(19) United States
(12) Patent Application Publication
(10) Pub. No.: US 2015/0030558 A1
(43) Pub. Date: Jan. 29, 2015

(54) SKINCARE COMPOSITIONS

(71) Applicant: Syrinx ZA Intenational Limited, London (GB)

(72) Inventors: Rupert Stephen Jude Bevan, Bath and North East Somerset (GB); Anthony Thomas Maleedy, Somerset (GB); Melinda Miriam Coss, Ste Sabine Born (FR)

(21) Appl. No.: 14/381,600

(22) PCT Filed: Feb. 26, 2013
(86) PCT No.: PCT/GB2013/050470

§ 371 Date: Aug. 28, 2014

(30) Foreign Application Priority Data
Mar. 1, 2012 (GB) 1203597.8

(51) Int. Cl. A61K 35/02; A61K 8/96; A61Q 5/02; A61K 36/185

(52) U.S. Cl. CPC A61K 35/02; A61K 36/185; A61K 8/965; A61Q 5/02
USPC 424/74; 424/725; 510/119

(57) ABSTRACT

The present disclosure relates to compositions. More particularly, the present disclosure relates to skincare composition. The present disclosure also relates to methods of forming extracts for inclusion in skincare compositions.

The present invention relates to compositions. More particularly, the present invention relates to skincare composition. The present invention also relates to methods of forming extracts for inclusion in skincare compositions.

Compositions applied to the skin, e.g. emollients, moisturising lotions (e.g. E45™ cream as currently sold in the UK by Reckitt Benckiser™), body butters, balms and shampoos (which are applied to hair but also contact the skin), for moisturisation of the skin generally, include ingredients which act to prevent irritation of the skin and generally moisturise the skin.

As described in N. P. O. Green et al., Biological Science 1 & 2, 2^{nd} edition, Cambridge University Press, 1990, pages 678-680, the term skin relates to the outer covering of vertebrate animals. The skin is composed of two main layers, the epidermis and the dermis. The epidermis is composed of many layers of cells forming a stratified epithelium. The skin includes pores, sweat glands and hair follicles. The outermost epithelial cells are continually being shed as a result of, for example, friction. Shed epithelial cells are a component of dust.

In the every day environment, particularly in urban and industrial environments, skin comes into contact with a number of potential irritants and/or pollutants. Common skin complaints include cracked skin and/or itchiness and/or weeping and/or rashes associated with skin complaints and diseases such as eczema, dermatitis, acne, psoriasis, athlete's foot, and allergies. Skin complaints can be exacerbated when skin comes into contact with environmental contaminants and/or pollutants, e.g. metal ions in the air and/or the dissolution of nickel from wrist watches and/or other jewellery into moisture on the skin.

It is common for persons suffering from cracked skin to apply moisturisers and/or emollients to their skin with a view to keeping the skin supple and thereby reducing the chance of cracking and/or flaking. Moisturisers and/or emollients generally tend to increase the skin's hydration by reducing evaporation, i.e. forming a layer over the epidermis so that water and other volatile compounds cannot evaporate from pores in the skin.

As known moisturisers and/or emollients generally form a layer over the epidermis which is substantially impermeable to water, they trap other potentially harmful pollutants from the air on the skin, e.g. on the outer epidermal layer or in pores. Thus, whilst known moisturisers and/or emollients can be beneficial in moisturising skin, they prevent the loss of the outer layer of the epidermis and thereby act to keep potentially hazardous materials, e.g. metal ions, adjacent and/or in the epidermis. This trapping of potentially hazardous materials, e.g. metal ions, is problematic because the potentially hazardous materials, e.g. metal ions, can enter the bloodstream and/or act to further inflame areas of skin.

Sapropel has previously been disclosed in PCT/GB2005/000226 to be used in generally raw forms, i.e. either having been dried by convection and then roasted or air dried and subsequently ground, in soap. Sapropel was, in that case, described as being used in combination with glycerine in the soap because the combination was found to be synergistic.

Sapropel is a clay-like material, which is known as a source material for oil and natural gas. The term, sapropel, is derived from the Greek sapros, meaning “decayed” and pelos meaning “mud”, and denotes a range of marine and lacustrine sediments containing organic and inorganic components. Sapropels range from the black organic oozes associated with the Silurian rock formations to variously coloured Holocene deposits.

Deposits of sapropel are mainly associated with sub-boreal lakes of Northern Europe, Siberia, Canada and the northern states of the U.S.A. Within Europe there are concentrations of sapropel-rich lakes in Karelia, Estonia, Latvia, Lithuania, Poland and the Czech Republic. Smaller amounts are reported to exist in Denmark, Finland, Sweden, the Netherlands, northern Italy and eastern parts of Germany. Extensive deposits are also found in the Russian Federation, Belarus and Ukraine.

As will be appreciated, not all sapropels are found as lake deposits. They may have their origin in peat formed in subsequent layers of vegetation. For example, sapropel from the Lake Sakhtysh region of north-west Russia is mined from beneath dry peat land.

Marine sapropels can also occur which are also Holocene. They are associated with the seas bordering arid regions, such as Namibia and the Sierra Nevada of Venezuela, and the eastern Mediterranean and Black Sea in Europe.

Tabulated below is a list of countries and regions of the world where sapropel is reported to be found, together with a description of geological age.

TABLE 1
Table 1: Countries and regions of the world where sapropel
is reported to be found, together with description
of geological age. Source: Andersons (1996).

| Continent | Type of deposit |
|---|---|
| Northern Europe: | |
| Finland | Lacustrine Quaternary |
| Sweden | ″ |
| Estonia | ″ |
| Latvia | ″ |
| Lithuania | ″ |
| Denmark | ″ |
| Netherlands | ″ |
| Baltic Sea | Marine Quaternary |
| Central Europe: | |
| Czech Republic | Lacustrune Quarternary |
| East Germany | ″ |
| Poland | ″ |
| Northern Italy | ″ |
| Romania | ″ |
| Southern Europe: | |
| Mediterranean Sea | Marine Silurian - Quarternary |
| Black Sea region | ″ |
| CIS: | |
| Belarus | Lacustrine Quaternary |
| Ukraine | ″ |
| Russia | ″ |
| Kaleria | ″ |
| Siberia: Omsk | ″ |
| Yakutsk | ″ |
| Nizhny | ″ |
| Novgorod | ″ |
| Tomsk | |
| The USA: | |
| Arkansas | Lacustrine Quaternary |
| Florida | ″ |
| Minnesota | ″ |
| Nebraska | ″ |
| Wisconsin | ″ |
| Canada | Lacustrine Quaternary |
| South America: | |
| Venezuelan coast | Marine Quarternary |
| Australia: | |
| Lake Cooroong | Lacustrine Quarternary |
| Africa: | |
| Namibia | Lacustrine Quaternary |

In the European regions, sapropels have been reported to form at a rate of 1 mm per annum. The organic components of sapropel accumulates in micro-laminations from a continuous rain of organic debris originating in vast reed beds bordering the lakes and is therefore autochthonous, i.e. originating from within the area of the lake. The inorganic component of sapropel is probably allochthonous, i.e. originating from outside the lake, but the migration of certain minerals such as silicon, calcium, magnesium and sulphur may originate from autochthonous organic sources.

Many sapropels are almost white-to-cream coloured. This reflects the amount of organic matter contained therein. As will be appreciated, as the organic component within the sapropel increases it will assume a darker colour; some sapropels are jet black.

Sapropels exhibit varying alkalinity. In this connection, sapropels having a pH greater than 7 are termed “lime-sapropels” and are usually characterised by the presence of several species of snails.

Sapropel can form in marine environments, as well as in freshwater lakes.

In marine environments, where the sea floor is too deep to allow oxygen to remain dissolved, sulphur-rich water acts as a reducing agent and provides an environment where organic debris can form sapropel. The sulphur itself is derived from the partial decomposition of plant and animal matter. In the areas of the sea beds where deposits of sapropel are found, the adjacent landmass is usually arid and well-leached of plant-growth supporting minerals. This may result in a correspondingly high supply of nutrients supporting a rich diversity of biota off the coast.

Typically, sapropel-rich lakes are situated on low-lying land. Generally, the lake bedrock is relatively insoluble and the lakeside soils tend to be podzols, from which nutrients are easily leached. As will be appreciated, the lakes themselves become sumps for these mobilised mineral salts, which are assimilated by reed beds that act as water-purifying agents. Sapropel forms on the lake floor in much the same way as peat forms on a raised or blanket bog. The organic compound is derived from limnic (surface) vegetation, in particular, reeds. As these herbaceous plants pass through their annual cycle of growth and decay, they give rise to a continuous stream of organic waste material that accumulates on the lakebed. Here decomposition is continued in the form of digestion of the lignified tissues. Sulphur from protein bonding is liberated in the form of hydrogen sulphide gas, which combines with dissolved oxygen to form soluble sulphurous acid. In a typical sapropel lake, there is little replacement oxygen as the water tends to be stagnant, and after a while, all the available oxygen is used up such that decomposition slows down, and eventually stops altogether. Thereafter, the digestion of organic material becomes anaerobically controlled, giving rise to chemical reductions and the precipitation of certain minerals.

Some lakes have been accumulating sapropel undisturbed for over 10,000 years. In some places, deposits of sapropel have displaced nearly all of the water. For example, Lake Zebrus in Latvia has approximately a half metre depth of water remaining.

As will be appreciated not all sapropel deposits are found in the lacustrine environment. For example, in the Lake Sakhtysh region of northern Russia, water has receded in recent time and some of the former lake land has undergone a succession to moss or reed beds, with a layer of peat formed above the sapropel deposit.

In the past, sapropel has been utilised as a fertiliser. In this connection, the use of sapropel as a fertiliser has not been pursued due to its low nitrogen content; this, despite the fact, that many attempts have been made to increase its nitrogen content. In addition, due to its mineral content, sapropel has also been utilised in some countries as a supplement to animal feed.

According to a first aspect of the present invention, there is provided a method of forming a skincare composition comprising a sapropel extract, the method comprising the steps of:
providing raw sapropel;
placing the raw sapropel in an alkali solution;
filtering the resultant solution to provide a solid and a filtrate of sapropel extract; and,
placing the filtrate of sapropel extract in a skincare composition.

Preferably, further comprising the step of concentrating the filtrate of sapropel extract prior to placing the filtrate of sapropel extract in a skincare composition; optionally, concentrating the filtrate of sapropel extract by removing 70-90%, or any intermediate value, of the water from the filtrate of sapropel extract.

Further preferably, wherein the step of concentrating the filtrate of sapropel extract comprises heating and/or stirring and/or placing under vacuum the filtrate of sapropel extract.

Advantageously, wherein the step of placing the raw sapropel in an alkali solution comprises placing the raw sapropel in a solution having a pH greater than 7.

Preferably, wherein the step of placing the raw sapropel in an alkali solution comprises placing the raw sapropel in a solution having a pH from greater than 7 to less than 14.

Further preferably, wherein the step of placing the raw sapropel in an alkali solution comprises placing the raw sapropel in a solution having a pH from greater than 8 to less than 12.

Advantageously, wherein the step of placing the raw sapropel in an alkali solution comprises placing the raw sapropel in a solution having a pH from greater than 9 to less than 11.

Preferably, wherein the step of placing the raw sapropel in an alkali solution comprises placing the raw sapropel in a solution having a pH from pH 9.5 to pH 10.5.

Preferably, wherein the step of placing the raw sapropel in an alkali solution comprises placing the raw sapropel in a solution having a pH of 10, 10.1, 10.2, 10.3 or 10.4.

Placing raw sapropel in a solution having a pH from pH 9.5 to pH 10.5 was found to be particularly advantageous. The use of lower pHs, for example from pH 8 up to pH 9, does not necessarily bring all of the humic extracts of sapropel into solution. By using a pH of from greater than 9.5 to less than 10.5, preferably 10, 10.1, 10.2, 10.3 or 10.4, substantially all humic extracts, along with fulvic extracts, vitamins, lipids, proteins and amino acids, are brought into solution so that filtration separates the solid matter of sapropel from filtrate of sapropel (sapropel extract) having beneficial properties.

Further preferably, wherein the step of placing the raw sapropel in an alkali solution comprises placing the raw sapropel in a solution of water and potassium hydroxide; and/or, ammonium hydroxide, sodium bicarbonate and/or sodium hydroxide.

According to a further aspect of the present invention, there is provided a skincare composition obtainable by any one of the above-mentioned methods.

According to a further aspect of the present invention, there is provided a skincare composition comprising a sapropel extract, the sapropel extract being obtainable by a method comprising the steps of:
providing raw sapropel;
placing the raw sapropel in an alkali solution; and,
filtering the resultant solution to provide a solid and a filtrate of sapropel extract.

Preferably, wherein the sapropel extract is obtained by a method comprising the steps of:
providing raw sapropel;
placing the raw sapropel in an alkali solution; and,
filtering the resultant solution to provide a solid and a filtrate of sapropel extract.

Further preferably, wherein the skincare composition is a moisturising cream, an emollient, a body butter, a balm, a shampoo, a hair conditioner, a pre-shampoo treatment, a hair lotion, a hair tonic, a shower gel or a liquid soap.

Advantageously, the skincare compositions of the present invention are for use in therapy.

Preferably, the skincare compositions of the present invention are for use in the treatment of any one or more of eczema, dermatitis, acne, psoriasis, athlete's foot, and/or allergies.

The previous combination of sapropel with glycerine in a soap utilised sapropel in a generally raw form.

The present inventors surprisingly discovered that processing raw sapropel in a particular way to provide a sapropel extract increases its moisturising capabilities and makes it suitable for inclusion in moisturising compositions applied to the skin, e.g. emollients, moisturising lotions, balms, body butters and shampoos.

On investigating the components of sapropel, the present inventors surprisingly discovered that sapropel contains high levels of humic and fulvic acids, together with vitamins, amino acids and lipids.

### Sapropel Extract

In order to prepare an extract of sapropel, raw sapropel (raw in the sense it had been taken from a naturally occurring sapropel deposit) was treated with a generally alkaline solution at a pH greater than 7; preferably, from greater than pH 7 to less than 14, or any intermediate pH; preferably, from greater than pH 8 to less than 12; preferably, from greater than pH 9 to less than 11; preferably, from pH 9.5 to pH 10.5; further preferably at pH 10, 10.1, 10.2, 10.3 or 10.4. A preferred alkaline solution was made up of potassium hydroxide and water, although any alkali material could be used, for example ammonium hydroxide, sodium bicarbonate and sodium hydroxide.

When the sapropel was subjected to the above described alkaline environment, the humic and fulvic acids, together with some lipids, vitamins, amino acids and sugars, present in the raw sapropel became soluble while much other organic matter present in the sapropel remained solid. The solid organic matter was filtered off and the remaining aqueous solution reduced down, e.g. by heating and/or evaporation under vacuum, to concentrate the humic and fulvic acids and other components. In a particular embodiment, 70-90% of the water present in the aqueous mixture was evaporated off, preferably, 70%, 71%, 72%, 73%, 74%, 75%, 76%, 77%, 78%, 79%, 80%, 81%, 82%, 83%, 84%, 85%, 86%, 87%, 88%, 89% or 90% of the water present in the aqueous mixture was evaporated off. This concentrated mixture of certain active ingredients from the sapropel was then used in the preparation of certain non-limiting embodiments of skincare compositions, as discussed below.

In a particular embodiment, raw sapropel was treated at a pH of 10, filtered and then the resultant aqueous mixture had 80% of the water present evaporated off. This produced a sapropel extract which was used in the formulations set out below.

### Moisturising Cream

A moisturising cream was prepared according to the following formulation protocol (the sapropel extract had been prepared as set out above):

| | Mass/g |
|---|---|
| Stage 1 | |
| Beeswax | 2 |
| Argan oil | 8 |
| Almond oil | 12 |
| Virgin coconut oil | 6 |
| Cetyl alcohol | 4 |
| Glyceryl stearate | 2 |
| Stage 2 | |
| Sodium stearoyl lactylate | 9 |
| Water | 140 |
| Sapropel extract | 1 |
| Glycerine | 5 |
| Optiphen ™ | 14 drops |
| Stage 3 | |
| Vitamin E oil | 2.4 |

All stage 1 ingredients were placed in a stainless steel vat and heated to between 75-85° C. so that the ingredients melted.

The water and the sapropel extract of the stage 2 ingredients were placed in a separate stainless steel vat and heated. Separately, the glyceryl stearate and the glycerine of the stage 2 ingredients were placed in a Pyrex™ jug and heated. The water and sapropel extract mixture, along with the Optiphen™, was then added over 5 minutes to the mixture of glyceryl stearate and glycerine, stirred and heated to 75-85° C.

The heated stage 1 ingredients were then added to the stage 2 ingredients and the mixture stirred for at least 5 minutes. The mixture eventually thickens; thickening can be speeded up by placing the vat containing the mixture in a cold water bath. When the temperature of the mixture decreased below 40° C., the stage 3 ingredient was added and stirred in.

The resultant moisturising cream had the consistency of double cream after preparation; within four days at standard temperature and pressure the cream thickened to the consistency of thick mayonnaise.

The moisturising cream was tested in an independent study by Cutest Systems Ltd. on twenty three volunteers. Each of the subjects suffered from atopic diathesis and the extent of their eczematous lesions was noted prior to application of the moisturising cream. A 96 hour patch test was carried out using the moisturising cream produced as set out above. The cream was applied to skin at 24 hour intervals. The cream was found, in all cases, not to cause irritation and was found to be well tolerated by the volunteers. In particular, a reduction in erythema was accompanied by a general reduction in lesions in eczema sufferers after the 96 hour patch test.

### Balm

A balm was prepared according to the following formulation protocol (the sapropel extract was prepared as set out above):

| | Weight % |
|---|---|
| Shea butter | 66 |
| Melon oil | 33 |
| Melaleuca alternifolia | 0.9 |
| extract | |
| Sapropel extract | 0.1 |

The shea butter was placed in a stainless steel vat and heated to between 75-85° C. for 15-30 minutes. The shea butter was then cooled to around 3-7° C., e.g. in a fridge and generally solidified. The shea butter and melon oil were then placed in a stainless steel bain marie and heated to around 50-60° C. until they melted. The sapropel extract and the Melaleuca alternifolia extract were then added and the resultant mixture stirred. The resultant mixture was then poured into sterile jars and left to set in a refrigerator at 0 to −10° C. for four hours.

The resultant balm had the appearance of a solid with a texture akin to beef dripping. The colour and texture was that of set honey.

The balm was tested in an independent study by Cutest Systems Ltd. on twenty three volunteers. The balm was found to be a good moisturiser and to act to reduce the symptoms of psoriasis.

### Shampoo

A shampoo was prepared according to the following formulation protocol (the sapropel extract was prepared as set out above):

| | Material | INCI | Weight % |
|---|---|---|---|
| Phase A | | | |
| 1 | Sapropel extract | | *5.00% |
| Phase B | | | |
| 2 | Water | Aqua | 59.62% |
| 3 | Jaguar C 162 (Rhodia) | Hydroxypropyl Guar | 0.30% |
| | | Hydroxypropyltrimonium | |
| | | Chloride | |
| Phase C | | | |
| 4 | Polyquaternium - 7 | Polyquaternium - 7 | 3.00% |
| 5 | MEA Lauryl Sulfate | MEA Lauryl Sulfate | 30.00% |
| 6 | Hydrotriticum WAA | Wheat Amino Acids | 0.30% |
| | (Croda) | | |
| 7 | Glycerine | Glycerin | 1.00% |
| Phase D | | | |
| 8 | Cocamide DEA | Cocamide DEA | 3.00% |
| 9 | Piroctone Olamine | Piroctone Olamine | 0.70% |
| | (Chemlink) | | |
| 10 | Fragrance: PF 67404 | Parfum | 0.50% |
| | Taupo | | |
| | (Phoenix Fragrances) | | |
| 11 | Argan Oil | Argania spinosa | 0.01% |
| | | (Argan) Oil | |
| Phase E | | | |
| 12 | Disodium EDTA | Disodium EDTA | 0.05% |
| 13 | ISOCIL PC | Methylchloroiso- | 0.07% |
| | | thiazolinone, | |
| | | Methylisothiazolinone | |
| 14 | Citric Acid | Citric Acid | q.s. pH 6.0 |
| 15 | Lauryl Betaine | Lauryl Betaine | 1.00% |
| 16 | Sodium Chloride | Sodium Chloride | q.s. |

(q.s. means quantitative)
The shampoo was prepared according to the following method:
Phase A. Disperse 1 into the water to be used in the product. Leave for 24 hours and then filter.
*Note:
The weight of this material is not included in the total %.
Phase B. Slowly add 3 into solution under high agitation. Once well dispersed add Citric Acid. The solution is clear. Continue to mix for 15-20 minutes.
Phase C. Slowly add 4 to the water and mix until dispersed. Mix in 5 to 7.
Phase D. Mix 9, 10 and 11 into 8. Slowly mix the resultant mixture (of phase D) into the overall mixture.
Phase E. Add 12 and 13 to the product. Adjust pH with 14 and add 15 to increase viscosity (and 16 if necessary) to finish.

The shampoo prepared according to the above had the following properties:

| Specifications: | |
|---|---|
| Appearance | Clear viscous liquid |
| Colour | Pale amber (natural) |
| Odour | As fragranced |
| pH | 6.00 |
| Viscosity @ 20 c. | 9,000 Spindle 3@10 rpm |

Applying the shampoo described above to twenty three volunteers, each of whom had flaky scalps, resulted in moisturisation of the scalp as well as a reduction in flaky skin on the scalp.

### Conditioner

A conditioner was prepared according to the following formulation protocol (the sapropel extract was prepared as set out above):

| | Material | INCI | % |
|---|---|---|---|
| Phase A | | | |
| 1 | Sapropel extract | | *5.00% |
| Phase B | | | |
| 2 | Water | Aqua | 86.58% |
| 3 | Cetearyl Alcohol | Cetearyl Alcohol | 4.50% |
| 4 | Behentrimonium | Behentrimonium | 1.50% |
| | Chloride (Varisoft | Chloride | |
| | BT 85 Pellets) | | |
| 5 | Cetrimonium Chloride | Cetrimonium Chloride | 1.50% |
| 6 | Glycerin | Glycerin | 2.00% |
| 7 | Olive Oil | Olea Europaea | 1.00% |
| | | (Olive) Oil | |
| 8 | Argan Oil | Argania spinosa | 0.50% |
| | | (Argan) Oil | |
| 9 | Disodium EDTA | Disodium EDTA | 0.05% |
| 10 | DC 949 | Amodimethicone (and) | 1.50% |
| | (Dow Corning) | Cetrimonium Chloride | |
| | | (and) Trideceth-12 | |
| Phase C | | | |
| 11 | Hydrotriticum | Wheat Amino Acids | 0.30% |
| | WAA (Croda) | | |
| 12 | Fragrance: PF | Parfum | 0.50% |
| | 67404 Taupo | | |
| | (Phoenix | | |
| | Fragrances) | | |
| 13 | ISOCIL PC | Methylchloroiso- | 0.07% |
| | | thiazolinone, | |
| | | Methylisothiazolinone | |
| 14 | Citric Acid | Citric Acid | q.s. pH 4.0 |

(q.s. means quantitative)
The conditioner was prepared according to the following method:
Phase A. Disperse 1 into the water to be used in the product. Leave for 24 hours and then filter.
*Note:
The weight of this material is not included in the total %.
Phase B. Heat the resultant mixture to 50° C. Add ingredients 3-10 into mixing vessel whilst stirring. Continue heating to approximately 70° C. or until the mixture is homogenous.
Phase C. Cool product to 35° C. then mix in 10-14. Continue to stir until the mixture is homogenous.

The conditioner prepared according to the above had the following properties:

| Specifications: | |
|---|---|
| Appearance | Opaque cream |
| Colour | White |
| Odour | As fragranced |
| pH | 4.00 |

Applying the conditioned described above to twenty three volunteers, each of whom had flaky scalps, resulted in moisturisation of the scalp as well as a reduction in flaky skin on the scalp.

### Pre-Shampoo Treatment

A pre-shampoo treatment was prepared according to the following formulation protocol (the sapropel extract was prepared as set out above):

| | Material | INCI | % |
|---|---|---|---|
| Phase A | | | |
| 1 | Sapropel extract | | 8.00% |
| 2 | Water | Aqua | 44.78% |
| Phase B | | | |
| 3 | Cetearyl Alcohol | Cetearyl Alcohol | 8.00% |
| 4 | Soy Oil | Glycine Soja | 20.00% |
| | | (Soybean) Oil | |
| 5 | Olive Oil | Olea Europaea | 3.00% |
| | | (Olive) Oil | |
| 6 | Argan Oil | Argania spinosa | 0.50% |
| | | (Argan) Oil | |
| 7 | Propylene Glycol | Propylene Glycol | 2.00% |
| 8 | Behentrimonium | Behentrimonium | 2.00% |
| | Chloride (Varisoft | Chloride | |
| | BT 85 Pellets) | | |
| 9 | Lanolin Anhydrous | Lanolin | 1.50% |
| | USP Azelis. | | |
| | code: 127200 | | |
| 10 | Cetrimonium Chloride | Cetrimonium Chloride | 1.80% |
| 11 | Glycerin | Glycerin | 4.00% |
| 12 | Disodium EDTA | Disodium EDTA | 0.05% |
| 13 | DC 949 | Amodimethicone (and) | 3.00% |
| | (Dow Corning) | Cetrimonium Chloride | |
| | | (and) Trideceth-12 | |
| 14 | Tocopherol | Tocopherol | 0.20% |
| Phase C | | | |
| 15 | Hydrotriticum WAA | Wheat Amino Acids | 0.60% |
| | (Croda) | | |
| 16 | Fragrance: PF 67403 | Parfum | 0.50% |
| | Shiel (Phoenix | | |
| | Fragrances) | | |
| 17 | ISOCIL PC | Methylchloroiso- | 0.07% |
| | | thiazolinone, | |
| | | Methylisothiazolinone | |
| 18 | Citric Acid | Citric Acid | q.s. pH 5.0 |

(q.s. means quantitative)
The pre-shampoo treatment was prepared according to the following method:
Phase A. Disperse 1 into the water and mix well to disperse.
Phase B. Heat the water to 50° C. Add ingredients 3-14 into mixing vessel whilst stirring. Continue heating to approximately 70° C. or until the product is homogenous.
Phase C. Cool product to 35° C. then mix in 14-18. Continue to stir until the product is completely homogenous apart from some solid particles.

The pre-shampoo treatment prepared according to the above had the following properties:

| Specifications: | |
|---|---|
| Appearance | Viscous cream with small particles |
| Colour | Pale grey |
| Odour | As fragranced |
| pH | 5.00 |

Applying the pre-shampoo treatment described above twenty three volunteers, each of whom had flaky scalps, resulted in moisturisation of the scalp as well as a reduction in flaky skin on the scalp.

In a particularly preferred embodiment, the pre-shampoo treatment was first applied to hair, and then washed off. The hair was then washed with the shampoo, followed by a rinse with water. Then the conditioner was applied, followed by a rinse. Application of the three products in this manner resulted in good overall moisturisation of the scalp and a reduction in flaky skin on the subject's scalp.

From the above, in all cases marked improvements in skin condition after starting treatment with the moisturising formulations of the present invention were observed.

Without wishing to be bound by theory, it is believed that the sapropel extract of the present invention contains concentrated fulvic and/or humic acids which act as ligands in the chelation of harmful residues on and/or in the skin, e.g. metal ions. By forming chelates, it is believed that residues present on the skin, e.g. metal ions, can be removed from being in contact with the skin, thus reducing potential inflammation whilst at the same time still providing a moisturising and/or emollient effect.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

In the present specification “comprises” means “includes or consists of” and “comprising” means “including or consisting of”.

1. A method of forming a skincare composition comprising a sapropel extract, the method comprising the steps of:
placing raw sapropel in an alkali solution;
filtering the resultant solution to provide a solid and a filtrate of sapropel extract; and,
placing the filtrate of sapropel extract in a skincare composition.

2. The method of claim 1, further comprising the step of concentrating the filtrate of sapropel extract prior to placing the filtrate of sapropel extract in a skincare composition; optionally, concentrating the filtrate of sapropel extract by removing 70-90%, or any intermediate value, of the water from the filtrate of sapropel extract.

3. The method of claim 2, wherein the step of concentrating the filtrate of sapropel extract comprises heating, stirring, placing under vacuum, or a combination thereof to the filtrate of sapropel extract.

4. The method of claim 1, wherein the alkali solution has a pH greater than 7.

5. The method of claim 4, wherein the alkali solution has a pH from greater than 7 to less than 14.

6. The method of claim 1, wherein the alkali solution has a pH from greater than 8 to less than 12.

7. The method claim 1, wherein the alkali solution has a pH from greater than 9 to less than 11.

8. The method of claim 1, wherein the alkali solution has a pH from pH 9.5 to pH 10.5.

9. The method of claim 1, wherein the alkali solution has a pH of 10, 10.1, 10.2, 10.3 or 10.4.

10. The method of claim 1, wherein the alkali solution comprises a solution of water and a base selected from the group consisting of potassium hydroxide; ammonium hydroxide, sodium bicarbonate, sodium hydroxide and combinations thereof.

11. A skincare composition obtainable by the method of claim 1.

12. A skincare composition comprising: a sapropel extract, the sapropel extract being obtainable by a method comprising the steps of:
placing raw sapropel in an alkali solution; and,
filtering the resultant solution to provide a solid and a filtrate of sapropel extract.

13. (canceled)

14. The skincare composition of claim 12, wherein the skincare composition is a moisturizing cream, an emollient, a body butter, a balm, a shampoo, a hair conditioner, a pre-shampoo treatment, a hair lotion, a hair tonic, a shower gel or a liquid soap.

15. (canceled)

16. A method for treating a skin condition of a subject, the method comprising:
administering to the subject an effective amount of the skincare composition according to claim 12 to treat a skin condition selected from the group consisting of eczema, dermatitis, acne, psoriasis, athlete's foot, and allergies.

17.-19. (canceled)
